(19) United States
(12) Patent Application Publication
(10) Pub. No.: US 2025/0303188 A1
(43) Pub. Date: Oct. 2, 2025

(54) LIGHT THERAPY DEVICE CONFIGURED FOR OPERATION IN WET ENVIRONMENTS

(71) Applicant: Alexander Gerard, Wayzata, MN (US)

(72) Inventor: Alexander Gerard, Wayzata, MN (US)

(21) Appl. No.: 19/092,731

(22) Filed: Mar. 27, 2025

Related U.S. Application Data
(60) Provisional application No. 63/570,666, filed on Mar. 27, 2024.

(51) Int. Cl. A61N 5/06

(52) U.S. Cl. CPC A61N 5/0616; A61N 2005/0626; A61N 2005/066; A61N 2005/0661; A61N 2005/0663; A61N 2005/0668

(57) ABSTRACT

A light therapy device capable of providing light therapy for a user which can be used in different environments, including wet environments, and which can provide differing levels and types of light therapy provides a valuable tool to provide therapy for a user. The therapy device may include a housing having a handle structure at a first end configured to be easily graspable by hand of the user, a light source contained within a main body and a plurality of flexible silicon fingers coupled thereto which are capable of transmitting light signals to a portion of the user's body. The ends of the flexible silicon fingers create a discontinuous treatment surface that can be placed adjacent the desired area of the user's body. The device further has charging capabilities to wirelessly charge an internal rechargeable power source, and has controls allowing for operation at various light levels.

### BACKGROUND

Red light therapy has been shown to provide several advantages for various purposes, including the treatment of several well know skin conditions and/or ailments. As an example, red light therapy can improve the appearance of skin, addressing the appearance of wrinkles, scars, redness and acne. Further, it is an accepted photodynamic therapy used to activate certain drugs to address skin cancer, psoriasis, acne, warts, and other types of cancers. The application of light therapy (including several portions of the spectrum) to provide beneficial treatments for other conditions are being discovered on a regular basis.

As will be appreciated, the configuration of light therapy devices in a manner which provides convenience, effectiveness, and flexibility is clearly beneficial. Users will be more likely to use such devices, thus getting desired treatment to a wide segment of the population. Ideally, the forms of light therapy can be applied or carried out in several ways, but if such treatments are not part of a user's traditional routine, it is less likely that they will be done. By making devices that can be incorporated into a daily routine, it is more likely that treatments will be carried out on a regular basis. Further, where light therapy treatments can be combined with other mechanisms, additional benefits can be achieved. Devices with the ability to do these things are not always readily available, and do not typically include all of the features required to provide convenience, effectiveness and flexibility.

### SUMMARY

The techniques and therapy device described herein provides for the treatment of a user's skin using light therapy. In one embodiment the device includes a housing having a handle structure at a first end configured to be easily graspable by hand of the user and a main body integral with the handle structure which is configured to contain a plurality of light sources. A plurality of flexible fingers are positioned adjacent the light sources in a manner to receive light signals received from the light sources and transmit the light signals to the user's body. The flexible fingers are generally transparent so that the light signals can be transmitted there through, and collectively form a discontinuous treatment surface that is easily positionable upon the desired treatment areas. The therapy device also has a rechargeable power source contained within the housing and operatively coupled to the light source to selectively provide power thereto. The housing further has a magnetic coupling element configured to cooperate with a related magnetic mounting structure thereby allowing the therapy device to be removably coupled to a support surface. Lastly, the therapy device is waterproof thus allowing for operation in a wet environment.

In some embodiments the flexible silicon fingers provide a predetermined spacing between the user's skin and the light sources. Additionally, the therapy device can include a controller operatively coupled to the therapeutic light sources, the rechargeable power source, the charging coil and the power switch, with the controller configured to control the light signals produced by the therapeutic light source, and to control the charging of the rechargeable power source.

### BRIEF DESCRIPTION OF THE DRAWINGS

Further advantages can be seen by reviewing the description below in conjunction with the drawings in which:

FIG. 1 is a view of the light therapy device in one intended environment;

FIG. 2 is a bottom perspective view of the light therapy device;

FIG. 3 is a top perspective view of the light therapy device;

FIG. 4 is a second bottom perspective view of the light therapy device;

FIG. 5 is a perspective view of the light therapy device and a related mounting structure;

FIG. 6 is an exploded view of the light therapy device and the mounting structure;

FIG. 7 is a side view of the light therapy device;

FIG. 8 is a top view of the therapy device;

FIG. 9 is a bottom view of the therapy device;

FIG. 10 is a bottom exploded view of one embodiment of a light therapy device; and

FIG. 11 is an upper exploded view of the light therapy device.

### DESCRIPTION

One embodiment of a light therapy device is further described below. This device is capable of use in multiple environments and capable of providing a combination of therapies to a user, including the treatment of dry skin, dealing with psoriasis and other skin conditions, helping to generate collagen, anti-aging, scar reduction, enhanced drug effectiveness, and other benefits. As discussed below, the device is capable of focused treatments to address localized conditions. It will be understood that certain variations and modifications are possible, and that such variations are within the scope intended by the inventors.

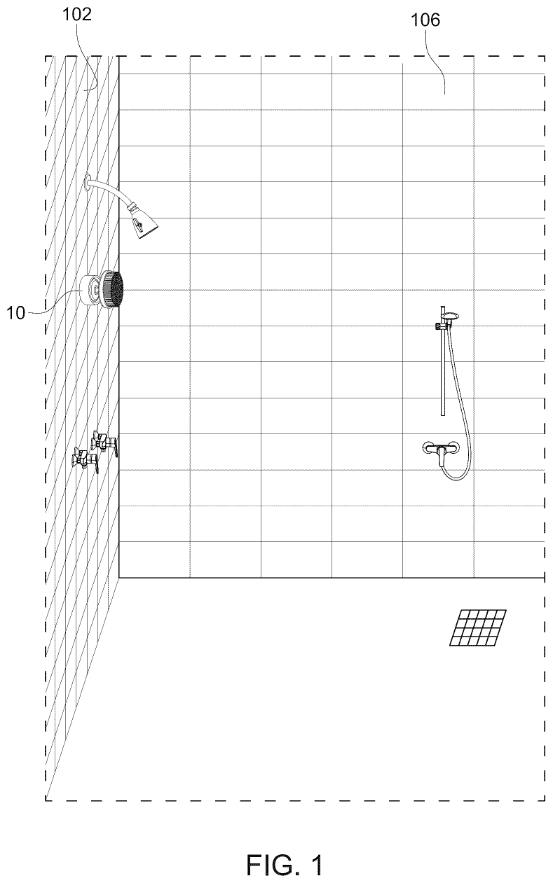

Referring to FIG. 1, a light therapy device 10 is illustrated in one intended environment 100. More specifically, the illustrated environment 100 is a shower, having multiple shower heads and related controls. In this example, light therapy device 10 can be mounted to one wall 102 in a manner to be accessible and useable by a user when in the shower. It will be anticipated that light therapy device 10 could also be utilized in several other environments, and the particular mounting location will be flexible depending on the desired needs and intended uses of the user.

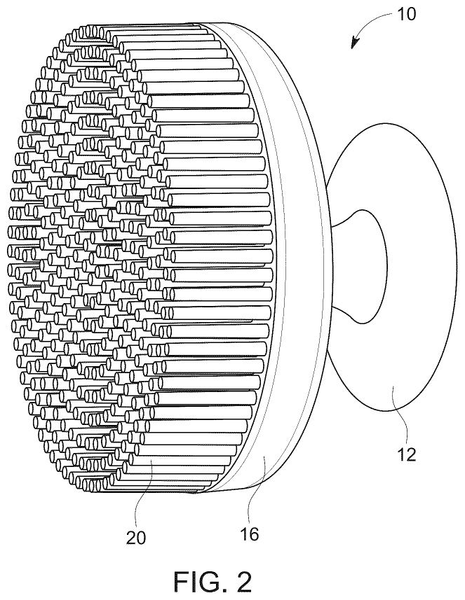

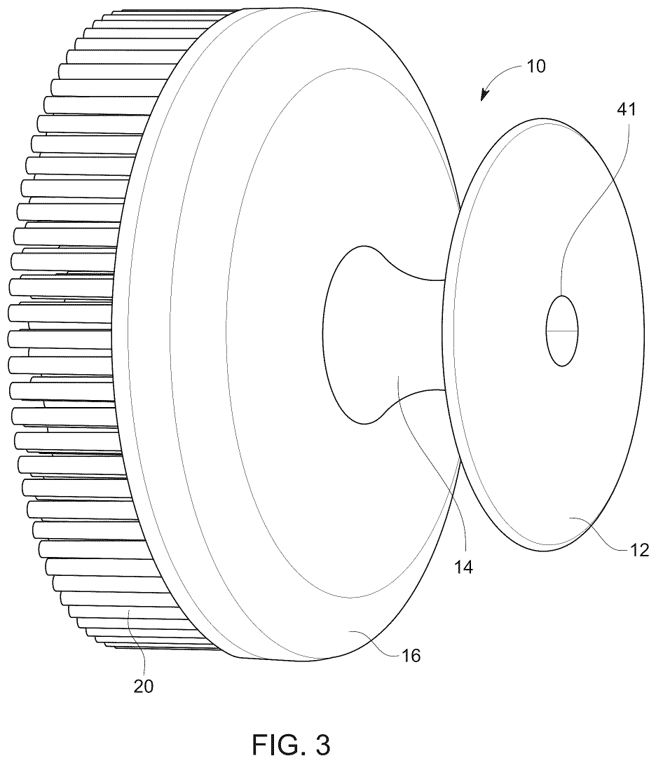

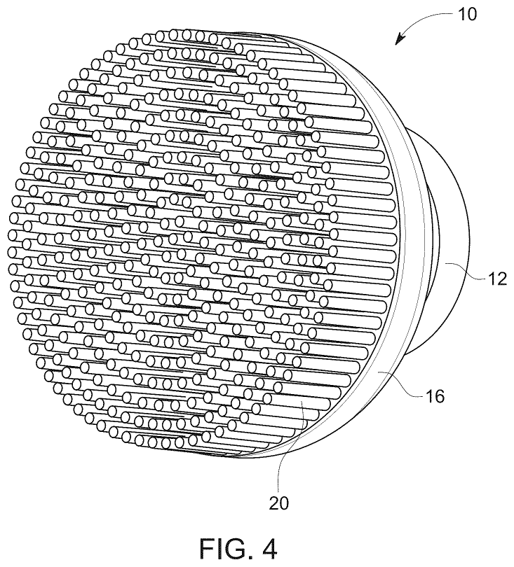

In FIGS. 2-4 various views of light therapy device 10 are presented, showing certain elements making up this embodiment. As can be seen, light therapy device 10 includes a first end 12 which is configured in a disc shape handle configuration. A power button 41 is positioned on a top surface of the first end 12 which is used to control the light therapy device 10. A neck portion 14 exists between first end portion 12 and a main body portion 16 situated at a second end of the light therapy device 10. An opposite side or lower side of main body portion 16 is configured to support a plurality of flexible fingers 20 which extend downwardly, and away from main body portion 16. As will be further discussed below, these flexible fingers 20 are specifically configured to provide a desired level of flexibility, while also being capable of transmitting light therethrough. Flexible fingers 20 are also capable of serving multiple functions, including light signal delivery, physical scrubbing/brushing, messaging, and setting a desired gap to regulate/creates a desired distance between an led board (contained within main body portion 16) and the user's skins surface.

In one embodiment, flexible fingers 20 are formed of silicone. As best seen in FIG. 2, the ends of silicone fingers 20 form a discontinuous treatment surface, which is configured in a manner to provide physical massage to portions of the user's body while light therapy is being applied. As will be contemplated, the stiffness of silicone fingers 20 can vary, depending on the type of massage therapy intended. It will be noted that light therapy device 10 is manufactured in a manner so that first end 12 (handle 12), neck portion 14 and main body 16 are over molded in a manner to provide water resistance. This is done in conjunction with the creation and formation of silicone fingers 20, but variations are possible. It is also noted that other structures and details could be contemplated, such as varying handle structures or body shapes, while all providing the combined therapies discussed herein. In alternative embodiments, the components of light therapy device 10 are configured so they can be coupled to one another in a water-tight manner, thus accommodating use in wet environments (such as the shower, as noted above). For example, the silicon fingers 20 could be coupled to a transparent plate or window member, with the transparent plate then coupled to the lower portion of main body 16. The transparent plate and the silicon fingers 20 would form a transparent therapy end of therapy device 10, which could be manufactured as a separate component and coupled to main body 16 in a manner that would achieve the desired waterproof characteristics.

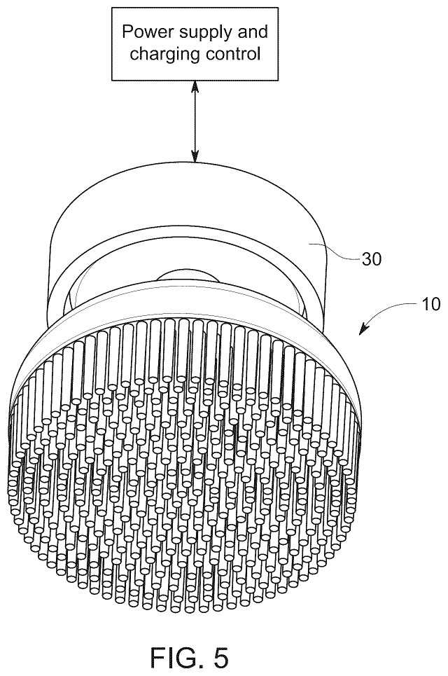

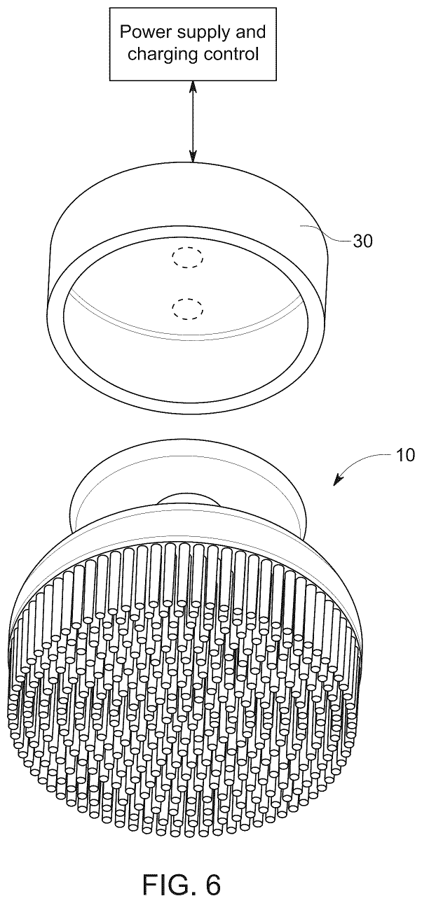

Turning to FIGS. 5 and 6, light therapy device 10 is illustrated along with a mounting structure 30, which is configured to allow easy connection and related removal of therapy device 10, when desired. Although not specifically illustrated in FIGS. 5 and 6, mounting structure 30 is intended to be attached to a supporting structure, such as a wall (e.g. a shower wall), a cabinet, a doorway, a door, or any other convenient surface. To accommodate attachment to such a structure, a pair of attachment holes 32 (shown in dashed lines) could be used for mounting. Alternatively, an adhesive tape or other types of adhesives could be used for attachment to an appropriate surface.

As will be discussed below, mounting structure 30 and therapy device 10 are configured to have a magnetic coupling system, providing for magnetic coupling between these two components. Again, in this embodiment therapy device 10 is configured to be over-molded in a manner that provides waterproofing so as to prevent contact between any liquids and related electronics contained within therapy device 10. By having a magnetic coupling system between mounting structure 30 and therapy device 10, the surfaces of both components can avoid unnecessary repetitive wear and tear as surfaces come into contact with one another. While a magnetic coupling mechanism is believed to be most efficient, several other coupling structures could be utilized as desired to best achieve the functions of therapy device 10.

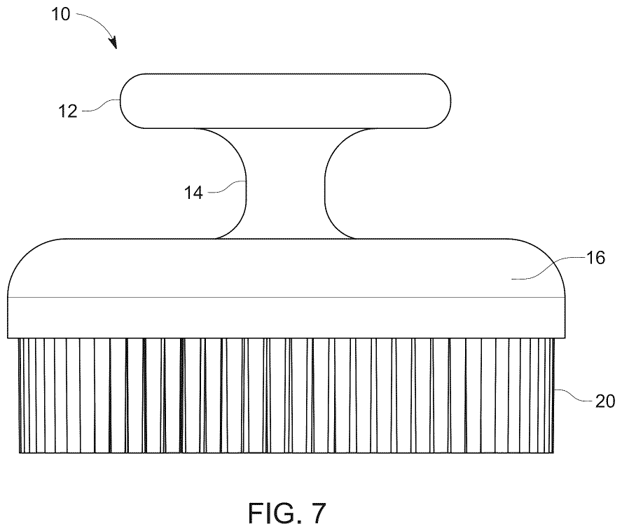

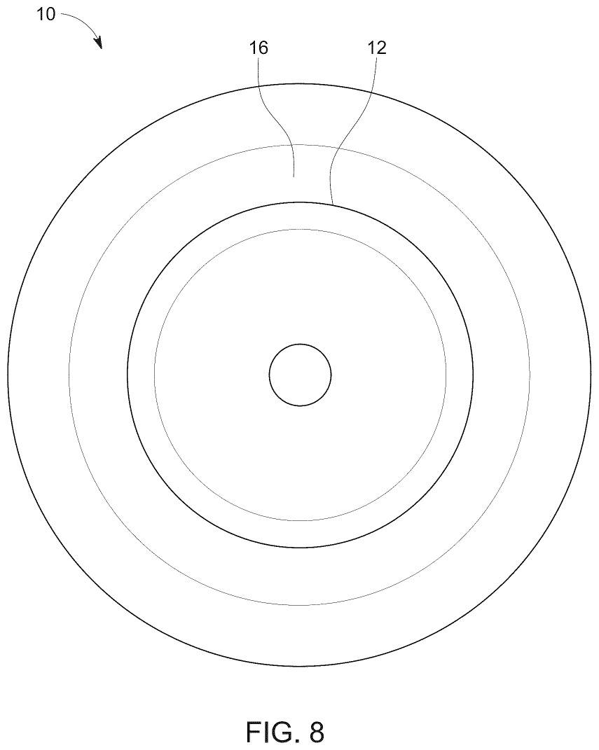

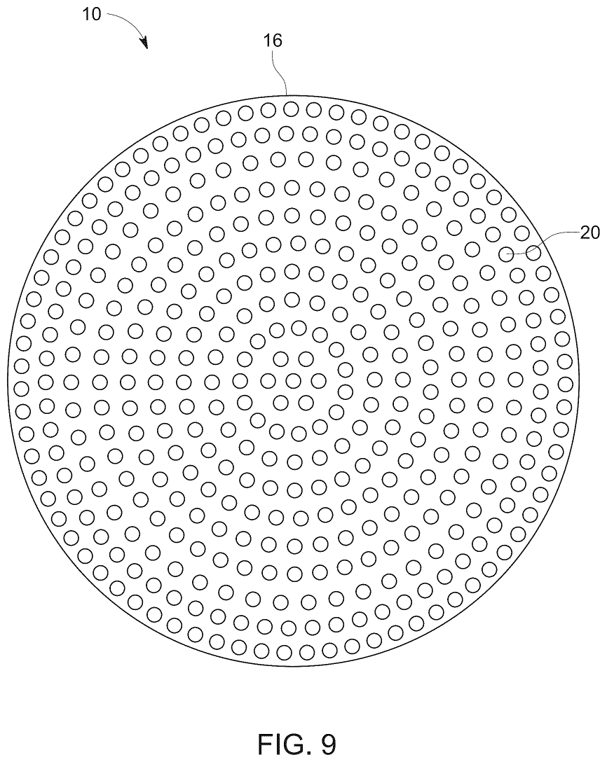

FIGS. 7-9 present side, top and bottom views of therapy device 10. As best shown in the side view of FIG. 7, handle portion 12 and neck 14 form a structure that is easily graspable by the user in many different ways. For example, the fingers of a user's hand could easily surround neck 14 and thus allow therapy device to be easily manipulated when applying light therapy to a portion of the user's skin. Given that handle portion 12, neck 14, and main body 16 are circular in nature (when viewed from the top or bottom), this allows the user to easily manipulate therapy device 10 so that light therapy can be applied to various portions of the user's body. The bottom view of FIG. 9 shows how the flexible fingers 20 are arranged in this embodiment. Those skilled in the art will recognize that this specific configuration could vary, so long as the ends of the flexible fingers 20 form a discontinuous treatment surface and are spaced away from the light source a desired distance.

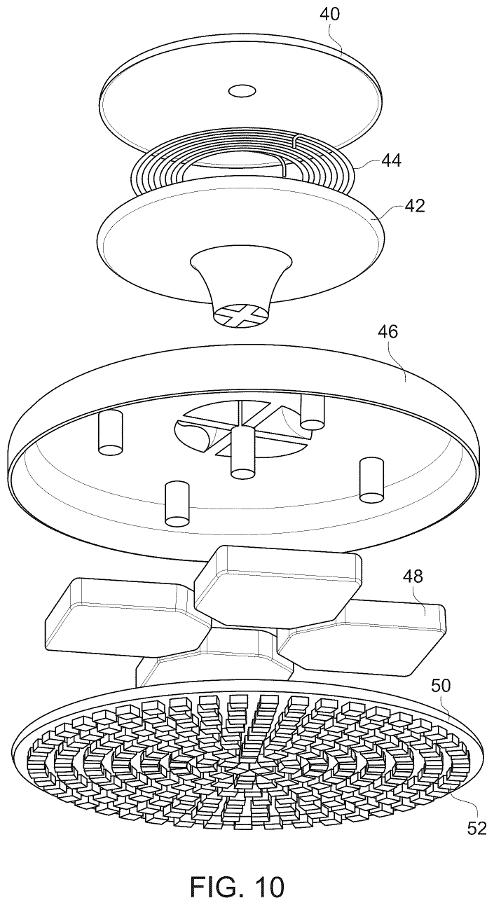

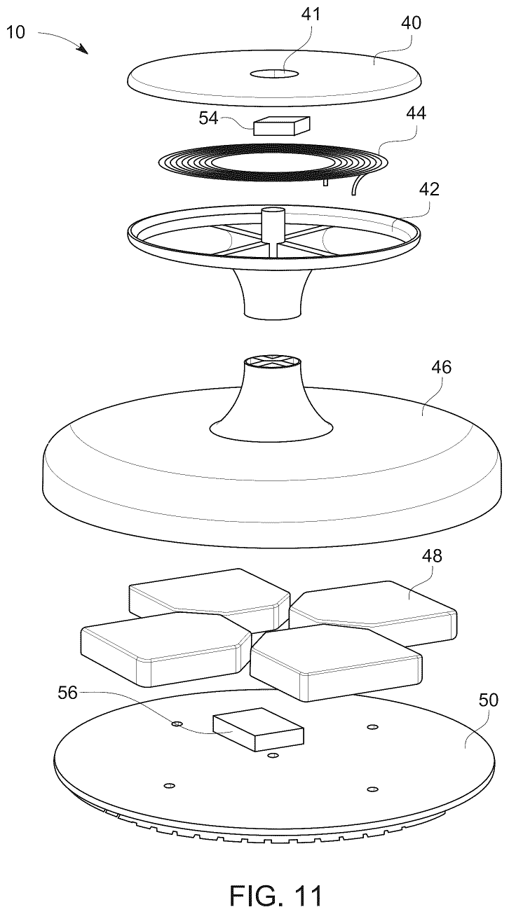

FIGS. 10 and 11 provide exploded views of the components making up therapy device 10. Here an upper cover 40 is designed to be coupled with an intermediate handle portion 42 to form handle portion 12. As shown, upper cover 40 includes a power button 41 which allows the user to control operation of the light therapy device 10. Between upper cover 40 and intermediate portion 42 an antenna structure 44 is situated, in a manner to allow for the receipt of a charging signal from a external source of power, thus allowing for the charging of rechargeable batteries contained within therapy device 10. Also situated between upper cover 40 and intermediate portion 42 is a switch mechanism 54 which will physically cooperate with power button 41 so operation of therapy device 10 can be controlled. Switch 54 could be a simple on/off type switch, or could be a multi-push switch allowing a user to select between various modes of operation (e.g. varied power levels, etc.). It will be appreciated that all wiring components and electrical connections are not necessarily illustrated in FIGS. 10 and 11, but those skilled in the art will appreciate that these components will exist in various formats and will also recognize where they could be positioned. Coupled to intermediate portion 42 is a main body housing 46. As shown, the mating portions of these two components create neck structure 14 discussed above. Contained within the main body housing 46 are a rechargeable power source 48 (here a plurality of batteries 48), which are positioned on an upper side of a circuit board 50. As also shown, main body housing 46 of this embodiment has an open lower end 47 which accommodate coupling of a therapy structure to main body housing in a manner to receive light signals.

Among other components, circuit board 50 will support a plurality of light sources 52 which could take on many different configurations. In some embodiments, light sources 52 will comprise LED's that are configured to produce the desired light signal(s). That is, each light source will be designed or configured to provide light signals of either a particular wavelength, or a variety of wavelengths. Additional control electronics, such as a controller or processor 56, will also be included and positioned at various locations on circuit board 50. Again, the open lower end 47 of main body housing 46 will allow these light signals to be transmitted in a desired manner. In another embodiment, the open lower end 47 will be covered by a window element (not shown) which allows the light signals to be transmitted therethrough. The window element will also serve as a support for the flexible fingers 20 in a manner that will allow for the light handling functions mentioned above to be accomplished.

Although not specifically illustrated as a particular component, it is contemplated that upper cap 40 will include a magnetic element. This may comprise either the element itself being magnetic or a metal plate that will interact with other components. This structure is configured to achieve desired magnetic coupling as discussed above. Further, it is contemplated that mounting device 30 will have a charging coil, or other type of charging structure that interacts with coil 44 shown in FIGS. 10 & 11.

As mentioned above, the light therapy device 10 may include several different light sources 52 which are configured to produce therapeutic light signals of different types. While red-light therapy has been mentioned above, it will be understood that light therapy device 10 is capable of operating at several different wavelengths, both selectively and in combination. In some embodiments, this will range from 200 nm to 1,400 nm, depending on the desired result. For example, it may be necessary to produce therapeutic light signals that are at selected wavelengths, or simultaneously at multiple selected wavelengths. For example, it may be beneficial to use 400 nm blue, 660 nm red, and 820 nm IR simultaneously. The combination of certain types of light signals at different power levels are known to provide certain therapeutic effects. For example, to generally increase collagen, the fingers could be configured and arranged so that 60 of them will deliver a red 630 nm signal, and another 60 will deliver an IR signal at 850 nm (with both sets powered at 20.1 W, with a radiant flux 4147 mw, and flux density of 9.22 mw/cm2.) As an alternative, an anti-aging and scar removal therapy could be created, using light sources 52 producing an IR signal at 1072 nm (radiant flux of 2973.46 mW, and flux density of 6.61 mw/cm2.) In another embodiment, therapy device 10 can be configured to address psoriasis and other skin diseases by have the LEDs produce a UVA signal at 415 nm, powered at 5 W (producing a radiant flux of 3744.43 mw and a flux density of 8.32 mw/cm2). Other possibilities include the use of 310 nm light signals for the treatment of psoriasis, and up to 1064 nm light signals for scar removal and anti-aging.

Again, flexible fingers 20 are configured to extend from the main body housing 16 in a manner that allows light signals to be emitted from an end portion thereof. In one contemplated use, flexible fingers 20 could be used to physically massage a portion of the user's skin while also providing therapeutic light signals. It will be recognized that therapy device 10 could be used for targeted treatments of localized conditions, as desired by the user. Alternatively, therapy device 10 could be configured to have a larger therapy delivery area to better provide whole body treatments.

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1. A therapy device for providing beneficial treatment to a user's skin, the therapy device comprising:
a housing having a handle structure at a first end configured to be easily graspable by a hand of the user, the housing further having a main body integral with the handle structure, the main body further having an opening at a second end opposite the first end;
a light source contained within the main body and positioned adjacent to the opening and configured to produce a therapeutic light signal;
a rechargeable power source contained within the housing and operatively coupled to the light source to selectively provide power thereto;
a charging coil positioned within the handle structure and operatively coupled to the rechargeable power source; and
a transparent therapy end coupled to the main body adjacent the opening in the housing, the transparent therapy end allowing the therapeutic light signal to pass therethrough, the transparent therapy end having a transparent base portion and a plurality of flexible silicon fingers coupled thereto, the plurality of silicon fingers further configured to allow the therapeutic light signal to pass therethrough and exit from an end portion of each of the plurality of silicon fingers;
wherein the housing further comprises a magnetic coupling element configured to cooperate with a related magnetic mounting structure thereby allowing the therapy device to be removably coupled to a support surface and which can be removed via a removal force from the user, and wherein the therapy device is waterproof thus allowing for operation in a wet environment.

2. The therapy device of claim 1 wherein the therapeutic light source has a wavelength between 200 nm and 1400 nm.

3. The therapy device of claim 1 wherein the plurality of flexible silicon fingers are configured such that the end portions thereof form a discontinuous massaging surface capable of simultaneously massaging a portion of the user's skin and providing light therapy thereto.

4. The therapy device of claim 1 wherein the flexible silicon fingers provide a predetermined spacing between the user's skin and the light sources.

5. The therapy device of claim 1 wherein the transparent therapy end further comprises a window member covering the opening and situated between the light source and the plurality of flexible silicon fingers.

6. The therapy device of claim 1 further comprising a power button at the first end configured to operably cooperate with a switch.

7. The therapy device of claim 6 further comprising a controller operatively coupled to the therapeutic light source, the power source, the charging coil and the switch, the controller configured to control the light signals produced by the therapeutic light source, and to control the charging of the rechargeable power source.

8. The therapy device of claim 7 wherein the switch and the controller are configured to allow a user to vary the light signal being produced by the therapeutic light source.

9. The therapy device of claim 8 wherein the therapeutic light source is capable of producing the light signal at a wavelength between 200 nm and 1400 nm.

10. The therapy device of claim 7 wherein the power switch is a multi-push switch configured to send a level signal to the controller, thereby allowing the controller to toggle between a plurality of operating modes, with each mode providing a light signal having a different wavelength.

11. The therapy device of claim 10 wherein the therapeutic light source is configured to simultaneously produce the light signal so as to have two different wavelengths.

12. The therapy device of claim 1 wherein the housing and therapy are formed from over-molding, thus causing the therapy device to be waterproof.

13. The therapy device of claim 1 wherein the housing, therapy end and button are all coupled to one another using a watertight seal.

14. A waterproof therapy device for providing light therapy to a desired treatment area of a user's skin, the waterproof therapy device comprising:
a housing having a handle structure at a first end configured to be easily graspable by hand of the user with a control button positioned upon an upper surface of the first end, the housing further having a main body coupled with the handle structure via a neck portion, the main body further having an opening at a second end opposite the first end;
a plurality of light sources configured to produce a therapeutic light signal, the plurality of light sources contained within the main body and positioned adjacent to the opening in the main body;
a rechargeable power source contained within the housing and operatively coupled to the plurality of light sources;
a charging coil contained within the handle structure and operatively coupled to the rechargeable power source;
a switch contained within the housing and positioned adjacent to the control button so that operating the control button causes interaction with the switch;
a controller contained within the handle structure and operatively coupled to the switch, the power source, the charging coil and the plurality of light sources to control operation of the therapy device;
a transparent therapy end coupled to the main body adjacent the opening in the housing, the transparent therapy end allowing the therapeutic light signal to pass therethrough, the transparent therapy end having a transparent base portion and a plurality of flexible silicon fingers coupled thereto, the plurality of silicon fingers further having a predetermined length and being configured to allow the therapeutic light signal to pass therethrough and exit from an end portion of each of the plurality of silicon fingers, wherein the end portions of the plurality of silicon fingers creates a discontinuous treatment surface that can be positioned adjacent the desired treatment area, and the length of the plurality of silicon fingers creates a desired separation between the desired treatment area and the plurality of light sources.

15. The waterproof therapy device of claim 14 wherein the housing further comprises a magnetic coupling element configured to cooperate with a related magnetic mounting structure thereby allowing the therapy device to be removably coupled to a support surface and which can be removed via a removal force from the user.

16. The waterproof therapy device of claim 15 wherein the plurality of therapeutic light sources operate at a wavelength between 200 nm and 1400 nm.

17. The waterproof therapy device of claim 16 wherein a first group of the plurality of therapeutic light sources operate at a first wavelength and a second group of the plurality of therapeutic light sources operate at a second wavelength.
